(19) United States
(12) Patent Application Publication
(10) Pub. No.: US 2024/0148384 A1
(43) Pub. Date: May 9, 2024

(54) FLOW DIVERTER DEVICES AND ASSOCIATED METHODS AND SYSTEMS

(71) Applicant: Narlin Beaty, Mt. Pleasant, SC (US)

(72) Inventor: Narlin Beaty, Mt. Pleasant, SC (US)

(21) Appl. No.: 18/548,902

(22) PCT Filed: Mar. 3, 2022
(86) PCT No.: PCT/US2022/018790

§ 371 Date: Sep. 3, 2023

Related U.S. Application Data
(60) Provisional application No. 63/156,345, filed on Mar. 3, 2021.

(51) Int. Cl. A61B 17/12; A61B 17/00

(52) U.S. Cl. CPC A61B 17/12118; A61B 17/00234; A61B 17/12036; A61B 17/12172; A61B 2017/00292; A61B 2017/00778; A61B 2017/00867; A61B 2017/00982; A61B 2017/12054

(57) ABSTRACT

A flow diverter device for restricting blood flow to an aneurysm at a blood vessel bifurcation is disclosed and described. The flow diverter device includes a linear device body that includes a high-density distal cap having an outer convex shape structurally configured to engage an aneurysm ostium on a blood vessel side at a blood vessel bifurcation, such that the high-density distal cap restricts blood flow into the aneurysm from the blood vessel bifurcation, a low-density section adjacent the high-density distal cap to allow blood flow through the blood vessel bifurcation, a stent section adjacent the low-density section and structurally configured to stabilize the linear device body in a lumen of the blood vessel bifurcation, and a proximal wire attachment at a lateral edge of the stent section, where the stent section terminally couples to a distal end of the proximal wire attachment.

### CROSS-REFERENCE TO RELATED APPLICATIONS

This application is a 371 U.S. Nationalization of International Patent Application No. PCT/US2022/018790, filed Mar. 3, 2022, which claims the benefit of U.S. Provisional Patent Application No. 63/156,345, filed on Mar. 3, 2021, each of which is incorporated herein by reference in its entirety.

### BACKGROUND

A cerebral aneurysm is a weak or thin spot on an artery in the brain that bulges out and fills with blood. Such an aneurysm can cause many health problems/risks, including putting pressure on neural tissue and rupture, which causes blood to spill into the surrounding tissue. A ruptured aneurysm can cause serious health problems such as hemorrhagic stroke, brain damage, coma, and even death.

Cerebral aneurysms, particularly those that are very small, do not bleed or cause other health problems, but often have the potential to do so if steps are not taken to curtail the bulging and weakening of blood vessel walls. These types of aneurysms are often detected during imaging tests for suspected neural problems or other medical conditions. Cerebral aneurysms can occur anywhere in the brain, but many form in the major arteries along the base of the skull.

### BRIEF DESCRIPTION OF THE DRAWINGS

FIG. 1 illustrates an aneurysm at a bifurcation of a blood vessel;

FIG. 2A illustrates a flow diverter device positioned within the bifurcation of a blood vessel at an aneurysm ostium with a high-density distal cap positioned at or against the blood vessel side of the aneurysm ostium in accordance with an example embodiment;

FIG. 2B illustrates a flow diverter device positioned within the bifurcation of a blood vessel at an aneurysm ostium with a high-density distal cap positioned in or within the aneurysm ostium in accordance with an example embodiment;

FIG. 3A illustrates a side view, a front view, and a back view of a flow diverter device in accordance with an example embodiment;

FIG. 3B illustrates a side view, a top view, a bottom view, a top-down isometric view, and a bottom-up isometric view of a flow diverter device in accordance with an example embodiment;

FIG. 4 illustrates a side view of a flow diverter device in accordance with an example embodiment;

FIG. 5 illustrates a side view of a flow diverter device in accordance with an example embodiment;

FIG. 6A illustrates a side view of a flow diverter device in accordance with an example embodiment;

FIG. 6B illustrates a side view of a flow diverter device in accordance with an example embodiment;

FIG. 7A illustrates a side view of an undeployed flow diverter device within a delivery device in accordance with an example embodiment;

FIG. 7B illustrates a side view of a deployed flow diverter device attached to a delivery device in accordance with an example embodiment;

FIG. 8A illustrates a view of an undeployed flow diverter device within a delivery device positioned within a bifurcated blood vessel at an aneurysm in accordance with an example embodiment;

FIG. 8B illustrates a view of a deployed flow diverter device attached to a delivery device positioned within a bifurcated blood vessel at an aneurysm in accordance with an example embodiment;

FIG. 9A illustrates a view of a cradle coupling for a flow diverter device in accordance with an example embodiment;

FIG. 9B illustrates a view of a flow diverter device released from a cradle coupling in accordance with an example embodiment;

FIG. 10A illustrates a top-down view of a cradle coupling in accordance with an example embodiment;

FIG. 10B illustrates an isometric view of a cradle coupling in accordance with an example embodiment;

FIG. 11A illustrates the release of a proximal wire attachment from a pusher in accordance with an example embodiment;

FIG. 11B illustrates the release of a proximal wire attachment from a pusher in accordance with an example embodiment; and

FIG. 11C illustrates the release of a proximal wire attachment from a pusher in accordance with an example embodiment.

### DESCRIPTION OF EMBODIMENTS

Although the following detailed description contains many specifics for the purpose of illustration, a person of ordinary skill in the art will appreciate that many variations and alterations to the following details can be made and are considered included herein. Accordingly, the following embodiments are set forth without any loss of generality to, and without imposing limitations upon, any claims set forth. It is also to be understood that the terminology used herein is for describing particular embodiments only, and is not intended to be limiting. Unless defined otherwise, all technical and scientific terms used herein have the same meaning as commonly understood by one of ordinary skill in the art to which this disclosure belongs. Also, the same reference numerals appearing in different drawings represent the same element. Numbers provided in flow charts and processes are provided for clarity in illustrating steps and operations and do not necessarily indicate a particular order or sequence.

Furthermore, the described features, structures, or characteristics can be combined in any suitable manner in one or more embodiments. In the following description, numerous specific details are provided, such as examples of layouts, distances, network examples, etc., to provide a thorough understanding of various embodiments. One skilled in the relevant art will recognize, however, that such detailed embodiments do not limit the overall concepts articulated herein, but are merely representative thereof. One skilled in the relevant art will also recognize that the technology can be practiced without one or more of the specific details, or with other methods, components, layouts, etc. In other instances, well-known structures, materials, or operations may not be shown or described in detail to avoid obscuring aspects of the disclosure.

In this application, “comprises,” “comprising,” “containing” and “having” and the like can have the meaning ascribed to them in U.S. Patent law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the composition's nature or characteristics would be permissible if present under the “consisting essentially of” language, even though not expressly recited in a list of items following such terminology. When using an open-ended term in this written description, like “comprising” or “including,” it is understood that direct support should be afforded also to “consisting essentially of” language as well as “consisting of” language as if stated explicitly and vice versa.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a given term, metric, value, range endpoint, or the like. The degree of flexibility for a particular variable can be readily determined by one skilled in the art. However, unless otherwise expressed, the term “about” generally provides flexibility of less than 1%, and in some cases less than 0.01%. It is to be understood that, even when the term “about” is used in the present specification in connection with a specific numerical value, support for the exact numerical value recited apart from the “about” terminology is also provided.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 1.5, 2, 2.3, 3, 3.8, 4, 4.6, 5, and 5.1 individually.

This same principle applies to ranges reciting only one numerical value as a minimum or a maximum. Furthermore, such an interpretation should apply regardless of the breadth of the range or the characteristics being described.

Reference throughout this specification to “an example” means that a particular feature, structure, or characteristic described in connection with the example is included in at least one embodiment. Thus, appearances of phrases including “an example” or “an embodiment” in various places throughout this specification are not necessarily all referring to the same example or embodiment.

The terms “first,” “second,” “third,” “fourth,” and the like in the description and in the claims, if any, are used for distinguishing between similar elements and not necessarily for describing a particular sequential or chronological order. It is to be understood that the terms so used are interchangeable under appropriate circumstances such that the embodiments described herein are, for example, capable of operation in sequences other than those illustrated or otherwise described herein. Similarly, if a method is described herein as comprising a series of steps, the order of such steps as presented herein is not necessarily the only order in which such steps may be performed, and certain of the stated steps may possibly be omitted and/or certain other steps not described herein may possibly be added to the method.

The terms “left,” “right,” “front,” “back,” “top,” “bottom,” “over,” “under,” and the like in the description and in the claims, if any, are used for descriptive purposes and not necessarily for describing permanent relative positions. It is to be understood that the terms so used are interchangeable under appropriate circumstances such that the embodiments described herein are, for example, capable of operation in other orientations than those illustrated or otherwise described herein.

As used herein, comparative terms such as “increased,” “decreased,” “better,” “worse,” “higher,” “lower,” “enhanced,” and the like refer to a property of a device, component, or activity that is measurably different from other devices, components, or activities in a surrounding or adjacent area, in a single device or in multiple comparable devices, in a group or class, in multiple groups or classes, or as compared to the known state of the art.

As used herein, the term “lumen” is used to refer to the internal space within a canal, duct, blood vessel, or like, within a subject. The term “lumen” can also refer to a tubular space in a catheter, a microcatheter, or the like in a device.

As used herein the term “proximal” refers to a location or point on a device that is closest to an operator as measured along a central axis of the device.

As used herein the term “distal” refers to a location or point on a device that is furthest from an operator as measured along a central axis of the device.

An initial overview of embodiments is provided below, and specific embodiments are then described in further detail. This initial summary is intended to aid readers in understanding the disclosure more quickly and is not intended to identify key or essential technological features, nor is it intended to limit the scope of the claimed subject matter.

Cerebral aneurysms can present a serious threat to patients, as they can enlarge and eventually rupture. Such a rupture can cause strokes, brain damage, and in some cases, death. The size, location, and type of the aneurysm can be a significant factor in the severity of the health risk to the affected patient.

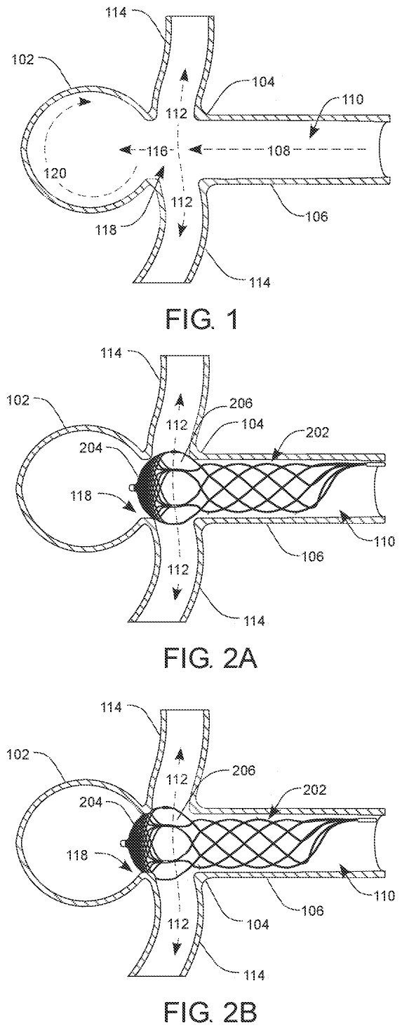

One type of aneurysm that can be challenging to effectively treat occurs at a bifurcation of a blood vessel. FIG. 1 shows such an aneurysm 102 at a bifurcation 104 of a blood vessel 106. Blood flows 108 through the lumen 110 of a primary blood vessel 106 and, in this example, splits to flow 112 through two secondary blood vessels 114. A portion 116 of the blood flow, however, flows into the aneurysm 102 through an aneurysm ostium 118 at the bifurcation 104. This portion 116 of the blood flow 108 can increase internal pressure and tends to circulate 120 around the aneurysm 102.

Various techniques exist to treat aneurysms at bifurcated blood vessels. One example of an invasive technique includes a surgical procedure involving placing a clip across the neck of the aneurysm to curtail blood from entering therein. An example of a minimally invasive technique involves placing a microcatheter within the aneurysm and deploying coils therein to cause thrombosis within the aneurysm to block blood flow. This technique, however, can puncture through the aneurysm wall and cause the aneurysm to rupture. Additionally, a portion of the coil can migrate out of the aneurysm and into the blood vessel, potentially causing damage to other blood vessels and/or neural tissue. Another example of a minimally invasive technique involves placing stents in the primary and secondary blood vessels to limit blood flowing into the aneurysm. Such a technique can be difficult to achieve and can significantly limit blood flow through the bifurcation of the blood vessel.

The present disclosure provides a minimally invasive technique using a flow diverter device that addresses many, if not all, of the aforementioned issues. It is noted, however, that while the following disclosure is directed to aneurisms at bifurcated blood vessels, it should be understood that such use is not limiting. As such, the present scope is intended to include any use for which the devices taught herein could be used, including any physiological vessel, duct, or the like, such as, for example, cardiac, peripheral, renal, hepatic, etc.

For example, as is shown in FIG. 2A, a flow diverter device 202 is positioned within the bifurcation 104 of the blood vessel 106 at the aneurysm 102. The flow diverter device 202 includes a high-density distal cap 204 positioned at or against the blood vessel side of the aneurysm ostium 118. As is shown in FIG. 2B, the high-density distal cap 204 of the flow diverter device 202 can be inserted into or within the aneurysm 102 against the lumen side of the blood vessel at the aneurysm ostium 118. In some examples, insertion of the high-density distal cap 204 into the aneurysm against the lumen side of the blood vessel can ensure that the parent blood vessels are open. Regardless of positioning, the high-density distal cap 204 reduces the blood flow entering the aneurysm 102, which is diverted to flow 112 through the secondary blood vessels 114. Additionally, the high-density distal cap 204 is sufficiently porous to allow some blood flow therethrough to facilitate endothelialization across the high-density distal cap 204, which will further block blood flow from entering the aneurism 102. This technique can significantly decrease the likelihood of aneurism rupture or other adverse cerebral events. It is noted that the depictions of the aneurysms and bifurcated blood vessels in FIGS. 1, 2A and 2B are merely simplified examples and should not be seen as limiting.

As such, flow diverter devices of the present disclosure generally reduce blood flow into the aneurysm from the blood vessel side of the bifurcation and divert the flow of blood from entering the aneurysm to the secondary blood vessels. Reducing such blood flow without the device physically entering the lumen of the aneurysm can significantly reduce the risk of aneurysm wall ruptures, which results in a significantly improved prognosis for the patient.

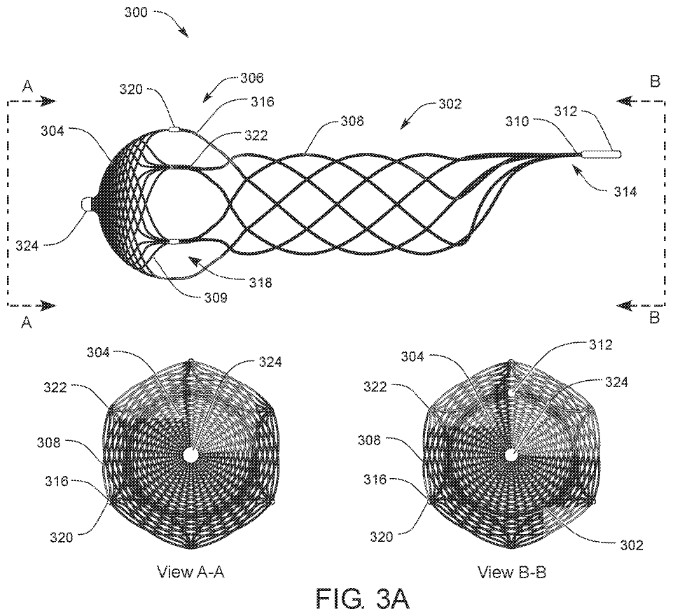

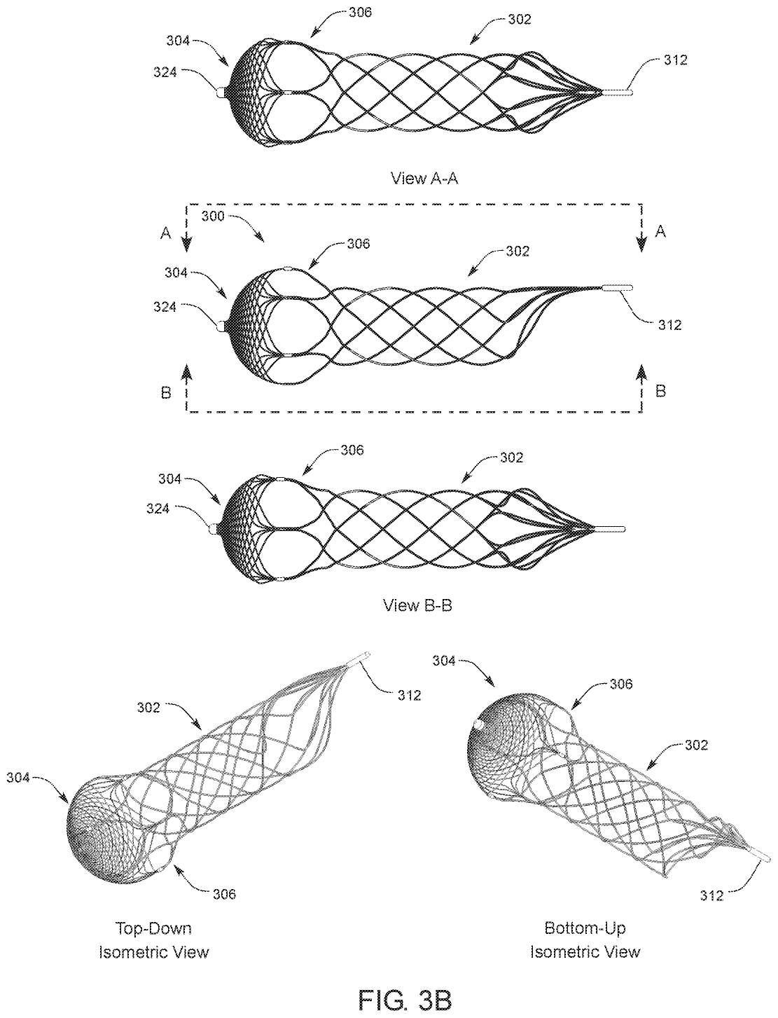

FIG. 3A shows one nonlimiting example of a flow diverter device of the present disclosure. The flow diverter device 300 includes a stent section 302, a high-density distal cap 304, and a low-density section 306 including multiple transverse openings 318. Flow diverter device 300, shown in FIG. 3A, can be made of any useful material capable of achieving results as outlined herein. For example, the flow diverter can be made from laser cut materials, polymeric materials, wire materials, carbon nanotubes, and the like, including combinations thereof. In one example, the flow diverter device 300 is made from braided wires 308, which allow the flexibility to design and make the different portions of the flow diverter device 300 to have different physical properties and functionality when deployed and placed inside of a blood vessel. In this example, the braided wires 308 converge 310 at their proximal ends and couple to a single proximal wire attachment 312, which can simplify the deployment of the flow diverter device 300. The braided wires can terminate at any number of proximal wire attachments, and thus the illustrated configuration should not be seen as limiting. In one example, the braided wires 308 are coupled to a single proximal wire attachment 312 laterally at the edge 314 of the stent portion 302.

Braided wires 308 can be braided in any useful pattern that is deployable and that is sufficiently stiff to hold the low-density section 306 and the high-density distal cap 304 in position at the aneurysm ostium. As the stent section 302 transitions distally into the low-density section 306, the braided wires 308 are brought together into bundles of braided wire, namely braided wire bundles 316, 322, which creates enlarged openings that effectively decreases the density of the low-density section 306 as compared to the stent section 302. The braided wire bundles 316, 322 can be secured or otherwise coupled or held together by any technique known to those skilled in the art. All of the braided wire bundles in a device can be secured by the same mechanism or different mechanisms. The example in FIG. 3A shows braided wire bundles 316, 322 having different securing mechanisms. Certain braided wire bundles 316 are secured together with wire clips 320 that crimp each braided wire bundle 316 securely together. Other braided wire bundles 322 lack wire clips and can be secured together with any technique capable of securing such braided wire bundles together. In one example, the braided wires of each braided wire bundle can be twisted or woven together. In another example, the braided wire bundles can be secured together by heat treatment. In yet another example, the braided wire bundles can be secured together with a bonding material. FIG. 3A also shows an example where the braided wire bundles 316, 322 separate and extend distally into distal braided wires 309, which are woven together to form the high-density distal cap 304. In one example, the distal braided wires 309 of the high-density distal cap 304 terminate and are coupled to a distal wire attachment 324.

View A-A of FIG. 3A shows a view of the flow diverter device 300 looking proximally from the distal end. This view shows one nonlimiting example of the weaving pattern of the braided wires 308 from the distal side of the high-density distal cap 304 as they converge and couple to the distal wire attachment 324. View A-A additionally shows the braided wire bundles 316, 320 and the wire clips 320 crimped at braided wire bundles 316. View B-B of FIG. 3A shows a view of the flow diverter device 300 looking distally from the proximal end. This view shows one nonlimiting example of the weaving pattern of the braided wires 308 from the proximal side of the high-density distal cap 304 as they converge and couple to the distal wire attachment 324. View B-B additionally shows the braided wire bundles 316, 320 and the wire clips 320 crimped at braided wire bundles 316. The stent section 302 and the proximal wire attachment 312 are also shown from this viewpoint.

FIG. 3B shows various views of the flow diverter device 300 including the stent section 302, the high-density distal cap 304, the low-density section 306, the distal wire attachment 324, and the proximal wire attachment 312. Additional reference numerals are not shown for the sake of clarity. View A-A of FIG. 3B shows a top-down view of the flow diverter device 300 where additional details of the weaving pattern of the braded wires through the stent section 302 and the low-density section 306 can be seen. View B-B of FIG. 3B shows a bottom-up view of the flow diverter device 300 where additional details of the weaving pattern of the braded wires through the stent section 302 and the low-density section 306 can be seen. Additionally, the top-down isometric view and the bottom-up isometric view show the weaving pattern of the braded wires through the stent section 302 and the high-density distal cap 304.

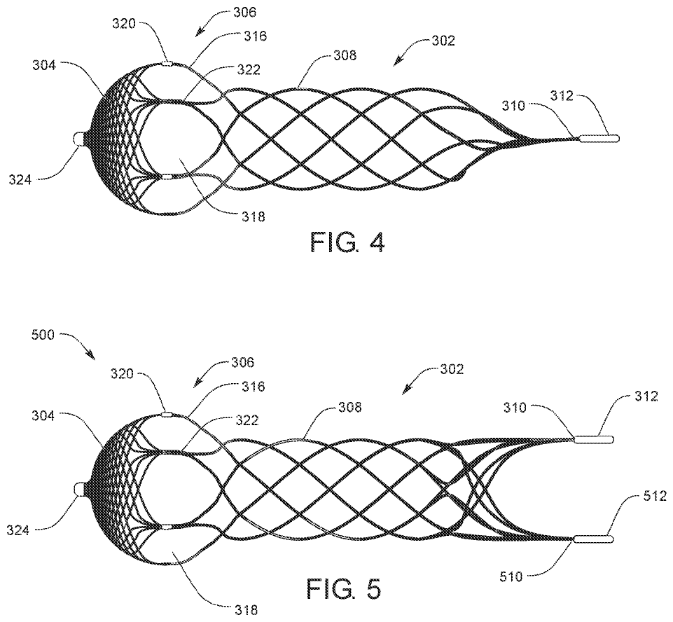

In another example, as is shown in FIG. 4, the braided wires are coupled to a single proximal wire attachment that is deployed and remains at the center of the primary blood vessel (or any other location therein), which also simplifies deployment of the flow diverter device due to the single proximal wire attachment. FIG. 5 shows another nonlimiting example of a flow diverter device of the present disclosure. Details of the flow diverter 500 in FIG. 5 having reference numerals as in FIG. 3 refer to the associated descriptions thereof. In this example, the braided wires 508 come together 310, 510 at two different points and are coupled to two different proximal wire attachments 312, 512.

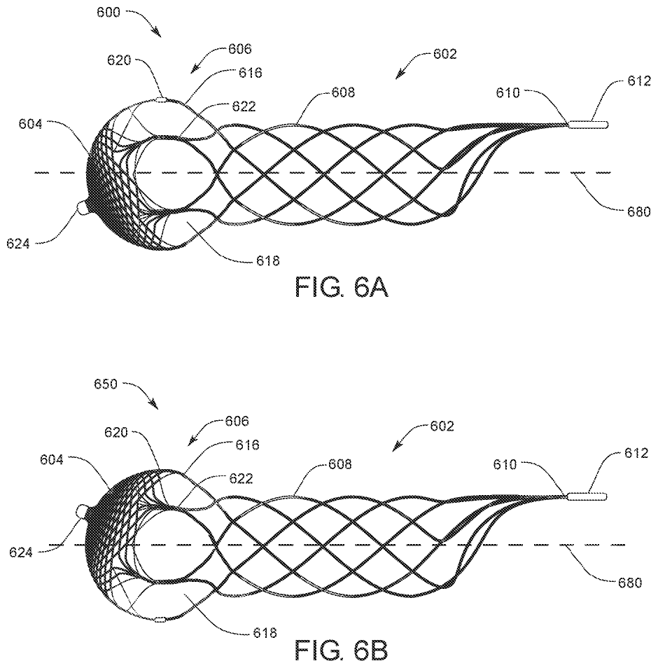

FIGS. 6A & 6B shows yet another nonlimiting example of a flow diverter device of the present disclosure. The flow diverter device 600, 650 includes a stent section 602, a high-density distal cap 604, and a low-density section 606. Flow diverter device 600, 650 shown in FIGS. 6A & 6B can be made of any useful material capable of achieving results as outlined herein. In one example, the flow diverter device 600, 650 is made from a braided wire 608, which allows the different portions of the flow diverter device 600, 650 to have different densities and thus different properties and functions when deployed and placed inside of a blood vessel. In this example, the braided wires 608 come together 610 and are coupled to a proximal wire attachment 612. The braided wires can terminate at any number of proximal wire attachments, and thus the illustrated configuration should not be seen as limiting. By coupling the braided wires 608 to a single proximal wire attachment 612 laterally at the edge 614 of the stent portion 602, however, deployment of the flow diverter device 600, 650 is simplified.

Braided wires 608 can be braided in any useful pattern that is deployable, that is sufficiently stiff to hold the low-density section 606 and the high-density distal cap 604 in position at the aneurysm ostium. As the stent section 602 transitions distally into the low-density section 606, the braided wires 608 are brought together into bundles of braided wire, namely braided wire bundles 616, 622, which creates enlarged openings 618 that effectively decreases the density of the low-density section 606 as compared to the stent section 602. The braided wire bundles 616 can be secured or otherwise coupled or held together by any technique known to those skilled in the art. All of the braided wire bundles in a device can be secured by the same mechanism or different mechanisms. The examples of FIGS. 6A & 6B show braided wire bundles 616, 622 having different securing mechanisms. Certain braided wire bundles 616 are secured together with wire clips 620 that crimp each braided wire bundle 616 securely together. Other braided wire bundles 622 lack wire clips and can be secured together with any technique capable of securing such braided wire bundles together. In one example, the braided wires of each braided wire bundle can be twisted or woven together. In another example, the braided wire bundles can be secured together by heat treatment. In yet another example, the braided wire bundles can be secured together with a bonding material. FIGS. 6A & 6B also show examples where the braided wire bundles 616, 622 separate and extend distally into braided wires 608, which are woven together to form the high-density distal cap 604. In one example, the braided wires 608 of the high-density distal cap 604 terminate and are coupled to a distal wire attachment 624. The distal wire attachment 624 and the surrounding weave of the braided wires 608 are offset or otherwise rotated away from the central axis 680 of the flow diverter device 600, 650. The distal wire attachment 624 and the surrounding weave of the braided wires 608 of the flow diverter device 600 in FIG. 6A are rotated upward toward the proximal wire attachment 312. In this case, the center of the dense portion of the high-density distal cap 604 is positioned at different orientation relative to the central axis 680 of the flow diverter device 600. The distal wire attachment 624 and the surrounding weave of the braided wires 608 of the flow diverter device 650 in FIG. 6B are rotated downward away from the proximal wire attachment 312. In this case, the center of the dense portion of the high-density distal cap 604 is positioned at different orientation relative to the central axis 680 of the flow diverter device 650. The rotation of the center of the dense portion of the high-density distal cap shown in FIGS. 6A& 6B allows the placement of the high-density distal cap at the ostiums of aneurysms that are offset from the central axis of the flow diverter device when positioned in the primary blood vessel. It is noted that, while the flow diverter devices of FIGS. 6A & 6B show this rotation toward and away from the proximal wire attachment, such is not limiting, and the rotation can be in any direction to accommodate the position of the aneurysm ostium relative to the central axis of the primary blood vessel.

In another example of the present disclosure, a flow diverter device can be made from mixed materials, or in other words, a combination of two or more of laser cut materials, polymeric materials, wire materials, braided wire materials, and the like, including any other materials known to those skilled in the art that can be beneficially used in the presently disclosed devices. In one example, a braided wire high-density distal cap can be coupled to a low-density section and a stent section where at least one of the low-density section or the stent section is made of a laser cut material. In another example, a braided wire high-density distal cap can be coupled to a low-density section of bundles of braided wire coupled to a stent section made of a laser cut material.

As has been described above, in one example the degree of porosity of the high-density distal cap can play a role in successfully diverting blood flow from an aneurysm over the long-term. If the porosity of the high-density distal cap is sufficiently low to block blood flow to a degree that thrombosis is facilitated on the aneurysm side of the high-density distal cap, a growing thrombus can spread through the periphery of the ostium of the aneurysm and across the structure of the flow diverter device. Such a thrombus can cause further complications to the patient that can, in some cases, be life-threatening. A higher porosity that diverts blood flow to the secondary blood vessels but which allows sufficient blood flow therethrough to facilitate fibrosis, endothelization, or delayed thrombosis across the high-density distal cap and the ostium of the aneurysm can result in a successful flow diverter device placement with significantly reduced complications. In terms of the flow diverter device, porosity is merely the inverse of the wire density of the high-density distal cap. Such can additionally be described as coverage when referring to the inverse of the porosity of the ostium with the flow diverter device in place (i.e., metal coverage for metal wires, polymer coverage for polymeric wires, etc.). One skilled in the art can readily ascertain a proper porosity/density of the high-density distal cap to achieve such a result, once in possession of the present disclosure. In one example, however, the density of the high-density distal cap can be from about 40% to about 85% or from about 50% to about 70%. In some examples the porosity of the high-density distal cap can be from about 15% to about 55% or from about 25% to about 45%. Furthermore, in some examples, the density of the braided wires in the high-density distal cap can vary from the center to the periphery. For example, the density can be highest at the center of the high-density distal cap where the braided wires couple to the distal wire attachment and lower at the periphery adjacent the low-density section. In one example, without limitation, the change from a higher density at the center of the high-density distal cap to a lower density at the periphery of the high-density distal cap can be a uniform transition. In another example, without limitation, the change from a higher density at the center of the high-density distal cap to a lower density at the periphery of the high-density distal cap can be a nonuniform transition.

The density of the high-density distal cap can be determined by the number, the diameter, and/or the weave pattern of the wires used in the device. As such, the number of wires and the number of wires in the wire bundles can vary, depending on the design and desired properties of the device. For example, the number of wires in a flow diverter device can be multiples of 3, 4, 5, 6, 7, 8, and so on, provided that the proper density of the resulting high-density distal cap can function as outlined herein. In one specific case, however, the number of wires is a multiple of 6, for example, 24 wires, 36 wires, or 48 wires, without limitation. As such, flow diverter devices would have 6 bundles of 4 wires or 4 bundles of 6 wires, 6 bundles of 6 wires, or 6 bundles of 8 wires or 8 bundles of 6 wires, respectively. As such, any weave pattern can be used that, taking into account the number of wires and wire bundles used, can be woven into a high-density distal cap having a uniform or nonuniform density as described and the desired density/porosity as understood by one skilled in the art. Furthermore, in one example, all of the wires can be the same length. In another example, at least a portion of the wires can have different lengths.

Furthermore, the wire used to create the wire bundles can be any physiologically compatible shape memory alloy capable of forming a flow diverter device as per the present disclosure. Nonlimiting examples of shape memory alloys can include Ag—Cd, Au—Cd, Co—Ni—Al, Co—Ni—Ga, Cu—Al—Be—X (where X is Zr, B, Cr, or Gd), Cu—Al—Ni, Cu—Al—Ni—Hf, Cu—Sn, Cu—Zn, Cu—Zn—X (where X is Si, Al, or Sn), Fe—Mn—Si, Fe—Pt, Mn—Cu, Ni—Fe—Ga, Ni—Ti, Ni—Ti—Hf, Ni—Ti—Pd, Ni—Mn—Ga, Ti—Cr or Ti—Nb, including combinations thereof. In another example, the wire can include a drawn filled tubing wire. While any combination of useful wire materials is contemplated, in one example the outer tube can be made of a nickel/titanium alloy and the inner core material can be a radiopaque material.

In one specific nonlimiting example, a metal alloy of nickel and titanium (Nitinol®) can be used as wires used to create the braided wire. Nitinol alloys are named according to the weight percentage of nickel in the alloy. For example, Nitinol 50, Nitinol 55, and Nitinol 60 include weight percentages of nickel in the alloy of 50%, 55%, and 60%, respectively. Any alloy of Nitinol can be used in the wire bundles that can be used to make a flow diverter device according to the present disclosure. Furthermore, the diameter of the Nitinol wire (or any other shape memory alloy wire) can be from about 0.008 inches to about 0.0005 inches in diameter in one example, from about 0.005 inches to about 0.0009 inches in diameter in another to example, and from about 0.002 inches to about 0.0015 inches in diameter, without limitation.

The stent section can have any weaving pattern of wire bundles, provided the stent section has sufficient longitudinal strength/stiffness to hold the high-density distal cap in position at the aneurysm ostium with sufficient radial force at the high-density distal cap to keep it in contact with the inner aneurysm ostium. Additionally, where a wire bundle crosses over other wire bundles, they can be woven in an over/under pattern, in one example. In other examples, the wire bundle can be woven in other patterns, such as two over one under and the like. Furthermore, in one example the wires in the wire bundles can be twisted around one another. In another example, the wires can be positioned side-by-side with little to no twisting. In another example, the wires can be positioned side-by-side with little to no twisting in certain locations along the stent section and twisted around one another in other sections. The same twisting examples can apply for the low-density section and the high-density distal cap.

For a Nitinol wire stent, the wire bundles can be heat treated such that the flow diverter device achieves a desired configuration once deployed at the aneurism ostium, or in other words, the flow diverter device rebounds to a fully expanded, deployed state. Additionally, such heat treatment can place the flow diverter device in a deployed position that matches a certain type or positioning of the aneurysm ostium relative to the primary blood vessel.

The distal wire attachment and the proximal wire attachment can be made from any useful physiologically compatible material capable of coupling to the wires of the braided wire bundles. In some examples, the distal wire attachment and/or the proximal wire attachment can be made of a radiopaque material to enhance visualization of the flow diverter device when in use. Furthermore, the wire clips that crimp together certain of the braided wire bundles can additionally be made of a radiopaque material in order to enhance visualization of the flow diverter section of the flow diverter device. The radiopaque material used for the proximal wire attachment, the distal wire attachment, and/or the wire clips can be any physiologically compatible material capable of coupling to the wires or wire bundles as per the present disclosure. Nonlimiting examples of radiopaque materials can include tantalum, tungsten, bismuth, gold, titanium, platinum, palladium, rhodium, iridium, tin, and mixtures, blends, composites, and alloys thereof. In another example, the proximal wire attachment, the distal wire attachment, and/or the wire clips can be made of a nonradiopaque material. In such cases, one or more radiopaque marker(s) can be coupled to the flow diverter device to allow visualization during placement.

In yet another example, the proximal wire attachment can additionally be utilized as a retriever for the flow diverter device. As the wires of the wire bundles are coupled to the proximal wire attachment, by pulling the proximal wire attachment back toward a delivery catheter, the wire bundles can fold back into the deliver catheter and the flow diverter device can be retrieved or partially retrieved. For example, the flow diverter device can be retrieved or partially retrieved for repositioning at the aneurysm ostium.

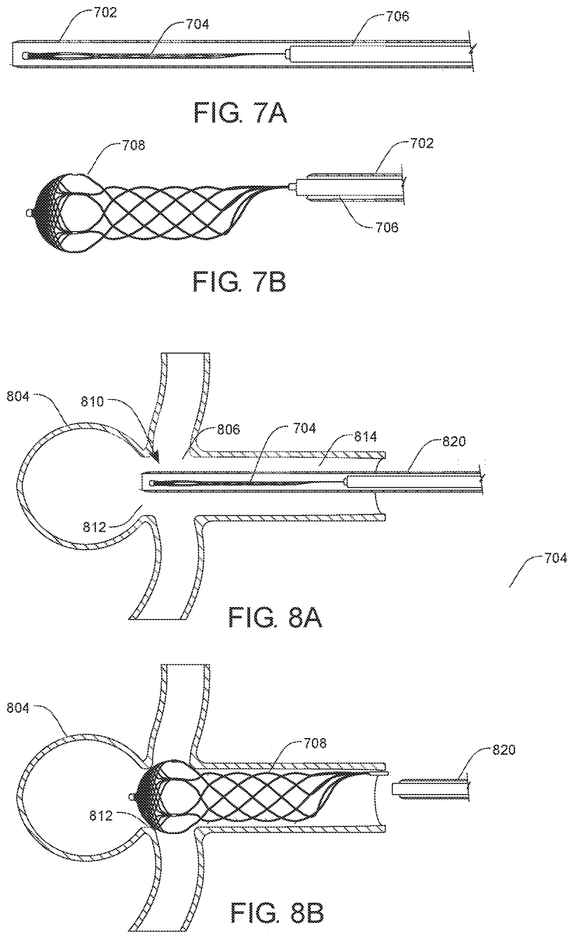

In another example, the present disclosure provides a system for delivering a flow diverter device, as is shown in FIG. 7A. Such a system can include a delivery catheter 702, an undeployed flow diverter device 704, and a delivery device 706. As the flow diverter device 704 exits the delivery catheter 702, as is shown in FIG. 7B, the flow diverter device is deployed as a deployed flow diverter device 708. It is noted that exit of the flow diverter device from the delivery catheter can include moving the flow diverter proximally, pushing the flow diverter device distally, or both. Following deployment, the deployed flow diverter device 708 can be released by the delivery device 706 (not shown). Release of the flow diverter device can be by any release mechanism that allows delivery, deployment, and release in a physiologically compatible manner. In one specific example, release can be achieved by electrolytic means. Once the flow diverter device is deployed and in position, electrolysis of the attachment mechanism can be electrically triggered, thus breaking down the electrolytic coupling between the flow diverter device and the delivery device.

FIGS. 8A & 8B show the placement and delivery of an undeployed flow diverter device 704 at an aneurysm 804 at a bifurcated blood vessel 806 by a delivery catheter 820. As is shown in FIG. 8A, the delivery catheter 820 is passed through the lumen of the blood vessel 814 and the distal end 810 of the delivery catheter 820 is positioned at or near the aneurysm ostium 812. As is shown in FIG. 8B, the flow diverter device 704 exits the delivery catheter 820 and is deployed as a deployed flow diverter device 708. It is noted that exit of the flow diverter device from the delivery catheter can include moving the flow diverter proximally, pushing the flow diverter device distally, or both. Once in position, the delivery catheter 820 can release the deployed flow diverter device 708.

Release of the flow diverter device can be by any release mechanism that allows delivery, deployment, and release in a physiologically compatible manner. In one specific example, release can be achieved by electrolytic means. Once the flow diverter device is deployed and in position, electrolysis of the attachment mechanism can be electrically triggered, thus breaking down the electrolytic coupling between the flow diverter device and the delivery device.

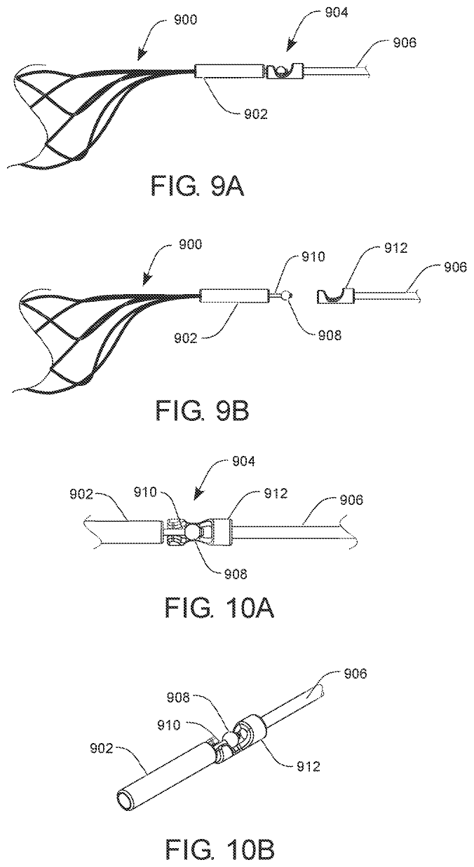

FIG. 9A shows an example of a release coupling for a flow diverter device 900. The flow diverter device 900 includes a proximal wire attachment 902 coupled thereto and a pusher 906. A cradle coupling 904 couples the proximal wire attachment 902 to the pusher 906. FIG. 9B shows a ball coupler 908 coupled to the proximal wire attachment 902 by a coupling spacer 910. FIG. 9B additionally shows a ball cradle 912 coupled to the distal end of the pusher 906. As such, the ball coupler 908 is positioned in the ball cradle 912 to create the cradle coupling 904.

FIG. 10A shows a top-down view and FIG. 10B shows an isometric view, both from the perspective of FIG. 9A, of the cradle coupling 904. The ball coupler 908 engages the ball cradle 912 to create the cradle coupling 904. The ball coupler 908 is thus secured against movement in a direction along the central axis of the pusher 906. The ball coupler 908 is released from the ball cradle 912 via movement through the open section of the ball cradle 912 in a direction away from the central axis of the pusher 906.

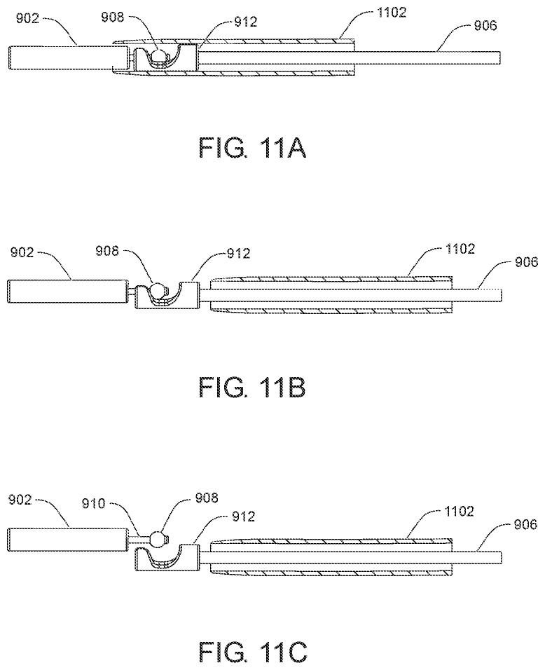

FIGS. 11A, 11B, and 11C provide a sequence of drawings showing the release of the proximal wire attachment 902 from the pusher 906. FIG. 11A shows the ball coupler 908 in the ball cradle 912 secured against movement along the central axis of the pusher 906 by the distal portion of the ball cradle 912. The ball coupler 908 is further prevented from being released from the ball cradle 912 by a sheath 1102 positioned over the ball coupler 908. FIG. 11B shows the sheath 1102 withdrawn from the ball coupler 908 and the ball cradle 912, freeing the ball coupler 908 to move through the opening of the ball cradle 912, thus affecting release of the proximal wire attachment 902 from the pusher 906, as is shown in FIG. 11C. It is noted, that while the sheath 1102 is in a position to prohibit release of the ball coupler 908 from the ball cradle 912, the proximal wire attachment 902 and the associated flow diverter device 900 can be withdrawn back into the delivery catheter by the pusher 906.

### EXAMPLES

The present disclosure provides, in one example, flow diverter device is provided, including a linear device body having an undeployed configuration and a deployed configuration, the linear device body sufficiently flexible to move through blood vessels in the undeployed configuration. When in the deployed configuration, the linear device body can further include a high-density distal cap having an outer convex shape structurally configured to be positioned adjacent or slightly within an ostium of an aneurysm at a blood vessel bifurcation, such that the high-density distal cap diverts blood flow from flowing into the aneurysm, instead redirecting blood flow to the blood vessel bifurcation, a low-density section adjacent the high-density distal cap structurally configured to allow blood flow through blood vessel bifurcation, and a stent section adjacent the low-density section and structurally configured to stabilize the linear device body in a lumen of the blood vessel bifurcation, wherein the high-density distal cap has a higher density of the braided wire compared to the stent section and the low-density section has a lower density of the braided wire compared to the stent section. The linear device body can additionally include a proximal wire attachment adjacent the stent section, wherein the high-density distal cap, the low-density section, and the stent section are substantially constructed of braided wire terminally coupled at a proximal end to the proximal wire attachment.

In another example, the flow diverter device can include a distal wire attachment coupled to distal ends of the braided wire.

In another example, the distal wire attachment is aligned along a central axis of the linear device body when in the undeployed configuration.

In another example, the distal wire attachment has an opening configured to allow passage of a wire from an inside region of the high-density distal cap to an outside region of the high-density distal cap.

In another example, the distal wire attachment is a radiopaque distal linear device body marker.

In another example, the proximal wire attachment is a radiopaque proximal linear device body marker.

In another example, the proximal wire attachment is positioned lateral to a central axis of the linear device body when in the deployed configuration and the braided wires from the stent section converge at the proximal wire attachment.

In another example, the flow diverter device can include a plurality of wire clips crimping together bundles of the braided wire to create the lower density of the braided wire.

In another example, the wire clips are radiopaque wire clip markers.

In another example, the braided wire is a shape memory braided wire.

In another example, the shape memory braided wire is a nickel-titanium alloy.

In another example, the braided wire is a drawn filled tubing wire.

In another example, the braided wire comprises a plurality of braided wire segments each extending from the proximal wire attachment to the distal wire attachment.

In another example, each of the plurality of braided wire segments includes wires of the same length.

In another example, the distal wire attachment is aligned along a central axis of the linear device body when in the deployed configuration.

In another example, the distal wire attachment is offset from a central axis of the linear device body when in the deployed configuration.

The present disclosure provides, in one example, a method for diverting blood flow from an aneurysm through a blood vessel bifurcation is provided. Such an example can include, positioning a delivery catheter containing the flow diverter device at an aneurysm of the blood vessel bifurcation, removing the delivery catheter from the flow diverter device to transition the flow diverter device from the undeployed configuration to the deployed configuration, such that the high-density distal cap of the flow diverter device is positioned at an ostium of the aneurysm to divert blood flow from entering the aneurysm.

In another example, the flow diverter device is repositioned to align the high-density distal cap with the ostium of the aneurysm, either during the transition from the undeployed configuration to the deployed configuration, following the transition from the undeployed configuration to the deployed configuration, or following at least partial retraction of the flow diverter device into the catheter.

The present disclosure provides, in one example, a delivery system for diverting blood flow from entering an aneurysm from a blood vessel bifurcation, comprising a delivery catheter including a flow diverter device contained therein, the delivery system configured to move through a system of blood vessels to a blood vessel bifurcation having an aneurysm and a delivery device releasably coupled to flow diverter device positioned in the lumen of the delivery device, the delivery device configured to maintain a position of the flow diverter device as the delivery device is removed proximally away from the flow diverter device.

What is claimed, is:

1. A flow diverter device, comprising:
a linear device body having a undeployed configuration and a deployed configuration, and, when in the deployed configuration, the linear device body further comprises;
a high-density distal cap having an outer convex shape structurally configured to engage an aneurysm ostium on a blood vessel side at a blood vessel bifurcation, such that the high-density distal cap restricts blood flow into the aneurysm from the blood vessel bifurcation;
a low-density section adjacent the high-density distal cap to allow blood flow through the blood vessel bifurcation;
a stent section adjacent the low-density section and structurally configured to stabilize the linear device body in a lumen of the blood vessel bifurcation; and
a proximal wire attachment at a lateral edge of the stent section, wherein the stent section terminally couples to a distal end of the proximal wire attachment.

2. The device of claim 1, wherein the low-density section includes a plurality of transverse openings.

3. The device of claim 1, wherein the high-density distal cap has a lower porosity compared to the stent section and the low-density section has a higher porosity compared to the stent section.

4. The device of claim 3, wherein the high-density distal cap has a porosity that allows sufficient blood flow into the aneurysm to inhibit thrombosis from forming on the aneurysm side of the high-density distal cap and that restricts sufficient blood flow into the aneurism to facilitate endothelialization on the high-density distal cap.

5. The device of claim 3, wherein the high-density distal cap has a porosity of from about 15% to about 55%.

6. The device of claim 3, wherein the high-density distal cap has a porosity of from about 30% to about 40%.

7. The device of claim 1, wherein the high-density distal cap and the low-density section are comprised of braided wire and the stent support is a laser cut stent support.

8. The device of claim 1, wherein the high-density distal cap, the low-density section, and the stent section are comprised of braided wire.

9. The flow diverter device of claim 8, wherein the proximal wire attachment is positioned lateral to a central axis of the linear device body when in the deployed configuration and the braided wires from the stent section converge at the proximal wire attachment.

10. The flow diverter device of claim 1, further comprising a distal wire attachment coupled to distal ends of the braided wire and aligned along a central axis of the linear device body when in the undeployed configuration.

11. The flow diverter device of claim 10, wherein the distal wire attachment includes a radioopaque material as a radioopaque distal marker.

12. The flow diverter device of claim 1, wherein the proximal wire attachment includes a radioopaque material as a proximal marker.

13. The flow diverter device of claim 1, wherein the braided wire is a shape memory braided wire.

14. The flow diverter device of claim 10, wherein the shape memory braided wire includes a nickel alloy.

15. The flow diverter device of claim 13, wherein the braided wire is a drawn filled tubing wire.

16. The flow diverter device of claim 8, wherein the braided wire comprises a plurality of braided wire segments each extending from the proximal wire attachment to the distal wire attachment.

17. The flow diverter device of claim 16, wherein each braided wire segment of the plurality of braided wire segments is the same length.

18. The flow diverter device of claim 16, wherein the braided wire increases in density from the periphery of the high-density distal cap to the distal wire attachment.

19. The flow diverter device of claim 16, wherein the distal wire attachment is aligned along a central axis of the linear device body when in the undeployed configuration.

20. The flow diverter device of claim 16, wherein the distal wire attachment is offset from a central axis of the linear device body when in the undeployed configuration.

21. A method for diverting blood flow from an aneurysm through a blood vessel bifurcation, comprising:
positioning a delivery catheter containing the flow diverter device of claim 1 at an aneurysm of the blood vessel bifurcation;
removing the catheter from the flow diverter device to transition the flow diverter device from the undeployed configuration to the deployed configuration, such that the high-density distal cap of the flow diverter device is positioned against an ostium of the aneurysm to divert blood flow from the aneurysm to secondary blood vessels of the blood vessel bifurcation.

22. The method of claim 21, wherein the flow diverter device is repositioned to align the high-density distal cap with the ostium of the aneurysm, either during the transition from the undeployed configuration to the deployed configuration, following the transition from the undeployed configuration to the deployed configuration, or following at least partial retraction of the flow diverter device into the delivery catheter.

23. A system for diverting blood flow from an aneurysm to secondary blood vessels of a blood vessel bifurcation, comprising:
a delivery device configured to move through a system of blood vessels to a blood vessel bifurcation;
the flow diverter device of claim 1 positioned in a lumen of the delivery device;
a guide wire releasably coupled to the proximal wire attachment of the flow diverter device and positioned in the lumen of the delivery device, the guide wire configured to maintain a position of the flow diverter device as the delivery device is removed from the flow diverter device.

24. The system of claim 21, wherein the delivery device is a delivery catheter.
